(19) United States
(12) Patent Application Publication
(10) Pub. No.: US 2011/0318776 A1
(43) Pub. Date: Dec. 29, 2011

(54) Method for identifying germs in a liquid medium

(76) Inventors: Xavier Nassif, Paris (FR); Agnès Ferroni, Clamart (FR); Brunhilde Dauphin, Paris (FR); Etienne Carbonnelle, Paris (FR); Jean-Luc Beretti, Les Clayes sous Bois (FR)

(21) Appl. No.: 13/138,520

(22) PCT Filed: Mar. 3, 2010
(86) PCT No.: PCT/IB2010/050913

§ 371 Date: Aug. 30, 2011

(30) Foreign Application Priority Data
Mar. 3, 2009 (FR) 09/00954

(51) Int. Cl. C12Q 1/04

(52) U.S. Cl. 435/34

(57) ABSTRACT

This invention relates to a method for identifying germs in a liquid medium, comprising adding a membrane detergent to the liquid medium containing components of a host infected with germs, i.e. cellular components and proteins from the extracellular environment, so as to release the germs from these components without degrading them, and in that the germs that have grown in the liquid medium are analysed by MALDI-TOF MS.

This invention relates to a method for identifying germs in a liquid medium.

A period of 24 to 48 hours is required to identify germs cultured in a biological fluid.

In fact, a culture of germs in a liquid medium must be subcultured on a solid medium.

This period of time is detrimental to the need to promptly initiate appropriate empirical antibiotic therapy before having the results of the antibiogram at hand.

Molecular biology techniques (PCR) applied directly to blood culture broths are faster; nevertheless, they take a minimum of about 3 h and require appropriate facilities and a degree of technical expertise.

WO 02/21108 (Large Scale Proteomics Corp. et al) relates to the detection and the characterization of micro-organisms comprising separating the micro-organisms from a mixture by 2D centrifugation.

The articles of Chen et al in Anal. Chem. 2008, 80,8694-8701 and J. Proteome Res; July 2007: 6(7); 2529-2538 disclose tension-active agents for mass spectrometry useful in proteomic.

The inventors' work focused on seeking means for overcoming the drawbacks of the prior art. The inventors sought to adapt the MALDI-TOF MS (Matrix-Assisted Laser Desorption/Ionisation Time-of-Flight Mass Spectrometry) technique to the identification of germs in a liquid medium, making the most of their expertise in both bacteriology and the analysis of microorganisms by mass spectrometry.

It is known that bacteria which have been grown on a solid medium can currently be identified by MALDI-TOF MS thanks to the specific profiles of species which have been obtained directly from colonies.

The inventors have found that this technique can be used by appropriately treating the germs contained in a liquid sample or biological sample, whether or not this sample has been enriched in a liquid medium.

Accordingly, the object of the invention is to provide a new method for identifying germs in a liquid medium.

The method of the invention is characterised in that a membrane detergent is added to the liquid medium containing components of a host infected with germs, i.e. cellular components and proteins from the extracellular environment, so as to release the germs from these components without degrading them, and in that the germs that have grown in the liquid medium are analysed by MALDI-TOF MS.

More particularly, the method of the invention comprises the steps of:
adding to the liquid medium a mild membrane detergent which does not damage the cell wall of prokaryotes, in order to separate the germs from the components of the infected host, in particular releasing intracellular germs, and to obtain spectra with reduced background noise, thus allowing easy identification of the germs;
recovering the germs from the liquid medium by differential centrifugation; and
analysing the germ pellet by MALDI-TOF MS, followed by comparing the results with appropriate germ databases.

This technique has the advantage of being rapid: it produces a gain in time of at least 24 h in the identification process by avoiding subculture on a solid medium, so that appropriate antibiotic therapy can thus be initiated without loss of time. Furthermore, the technique is easy to implement.

The mild membrane detergent is added to the liquid medium to a final concentration of at most 5%, more preferably of 0.5-5%, most preferably of 1%.

According to other conditions, the detergent is added to a final concentration of 1 to 5%, preferably 2.

Appropriate compounds include saponin, n-octyl glucoside, n-dodecyl-glucoside, octanoyl-N-methylglucamide, decanoyl-N-methylglucamide, n-dodecyl-β-D-maltoside.

Saponin is a preferred detergent.

Advantageously, contact time between the liquid containing the germs to be analysed and the detergent is at most 8 min, more preferably 1-8 min, more particularly approximately 5 min.

The mixture is centrifuged, advantageously after adding distilled water.

Appropriate centrifugation conditions are 20,000 rpm at most, particularly 10,000-15,000 rpm, for 15 min at most, preferably for approximately 1-15 min, and more particularly for approximately 2 min.

According to other conditions, the centrifugation is performed at 8000 t/minat most, preferably 3000-5000 t/min, during 15 min at most, preferably about 5-15 min, and more particularly during about 5 min.

The supernatant is then discarded and the pellet is taken up in distilled water and centrifuged, advantageously under the conditions defined above. The pellet is recovered and used for the MALDI-TOF MS analysis. The results obtained are compared with the databases.

Liquids used in the method according to the invention include, for example, blood cultures from patients positive for a particular germ, joint fluids inoculated or not in the operating room in blood culture vials and producing a culture positive for a particular germ, or any other biological fluid, e.g. urine, cerebrospinal fluid, drainage fluid from drains or abscesses, provided the infection is suspected to be monomicrobial. This can be detected either directly from the biological fluid, or after enrichment in a liquid medium when the inoculum is sparse.

The spectrum is advantageously analysed according to the technique described in international application WO2008/084409, filed on 8 Jan. 2008, for ‘Means for identifying a germ isolated from a clinical sample’.

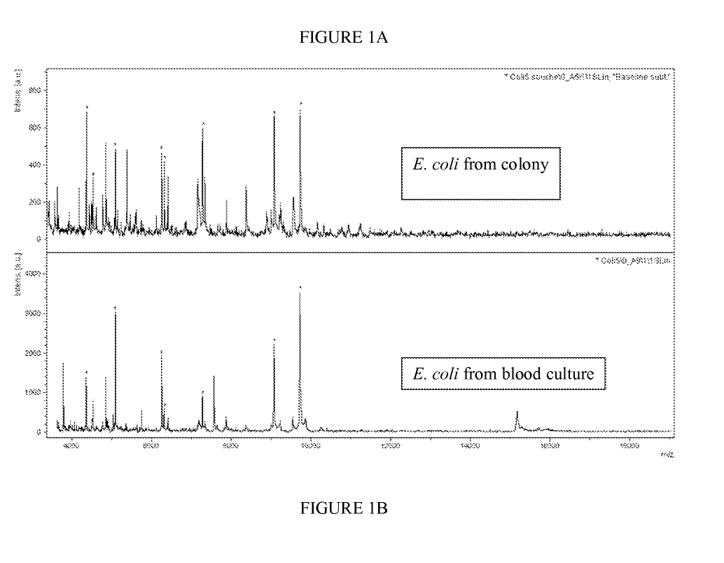

Various types of germs can be identified such as bacteria, yeasts, fungi or, if appropriate, filamentous fungi. Other features and advantages of the invention are set forth the following examples, in which reference is made to FIGS. 1A and 1B which are the MALDI-TOF MS graphs obtained for germs isolated from blood cultures and one isolated colony, respectively Common peaks between the two spectra are marked by “*”. These peaks are specific of the E. coli species (a.u: arb. unit (absorption)).

### MATERIAL AND METHODS

### Blood and Fluids Cultures

Preliminary tests used negative blood culture flasks without charcoal (bioMérieux, Marcy l'Etoile, France). They were artificially contaminated with 10⁴ cells of commonly isolated pathogens (table1), then placed in the automated blood culture apparatus Bact/Alert (bioMerieux, Marcy-l'Etoile, France) until detection of positivity. In addition, different pathological fluids from patients were tested: positive blood cultures (table 2 and 3) or different fluids spiked into blood culture flasks (table 4).

Two aliquots from the blood culture bottle were taken. The first aliquot was taken for MALDI-TOF-MS processing. The second was used for Gram staining, antibiotic suceptibility testing, and appropriate subcultures for microbiological identification using conventional microbiological techniques.

### MALDI-TOF-MS

Two hundred microliters of the positive blood culture broth (or 1 ml of enrichment liquids) were transferred into a plastic tube containing 40 μl (or 200 μl for enrichment liquids) of a solution of 5% saponin to release intracellular bacteria. After 5 minutes incubation, distilled water was added up to 1.5 ml and 2 consecutive washes in distilled water were performed at 16600 g for one minute. The supernatant was discarded and 5 μl of 10% trifluoroacetic acid was added to the pellet. One μl of this mixture was spotted (2 wells/sample) onto a MALDI sample target (Bruker Daltonics, Bremen, Germany) and allowed to dry at room temperature. One microliter of absolute ethanol was then added to each well, and the mixture allowed to dry. One μl of matrix solution DHB (2,5-dihydroxybenzoic acid, 80 mg/ml, 30% acetonitrile, 0.1% trifluoroacetic acid) was then added and allowed to co-crystallize with the sample. Samples were processed in the MALDI-TOF-MS spectrometer (Microflex, Bruker Daltonics) with the Flex Control software (Bruker Daltonics). Positive ions were extracted with an accelerating voltage of 20 kV in linear mode. Each spectrum was the sum of the ions obtained from 400 laser shots performed automatically on different regions of the same well. The spectra were analyzed in an m/z range of 3640 to 2000, and compared with those of a reference database (Andromas@SAS, Paris, France). This database has been engineered as previously described and encompasses the pathogens encountered in human pathology (1, 2). The identification of the tested strain corresponds to the species of the reference strain having the best match in the database. The analysis takes also into account the difference between the first two species having the best matches with the reference database. The species identification was considered to be valid if, for one of the two sample deposits, the percentage of matched peaks was at least 60% of that of the first species proposed in the database, after analysis by the Andromas@ software (Andromas SAS), and if the difference between the first two species having the best match in the database is at least 10%. If the latter condition was not fulfilled, the identification was considered as being correct at the level of the group/genus/family if the first two matches belonged to the same group/genus/family of bacteria. In all other cases, the results were considered as irrelevant. It should be pointed out that most unreliable identifications were due to poor quality spectra. When the blood cultures contained several bacterial species as seen on Gram staining, databases specific for Gram negative bacilli and/or Gram positive cocci were used.

### Results

### 1) Identification of Germs Spiked in Blood Culture Flasks

In order to determine whether an accurate identification of pathogens could be obtained from bacteria grown in liquid media, pilot experiments were first performed using blood culture bottles spiked with commonly isolated pathogens. FIGS. 1A and 1B show an example of spectrum obtained with Escherichia coli grown in blood culture bottles, compared to that of the same strain obtained from an isolated colony. A total of 292 bacterial strains and 20 Candida species were spiked in blood culture bottles. Results are shown in Table 1.

TABLE 1
Microbiological identification by MALDI-TOF-MS of blood cultures spiked with different species

| Species | | | | | | |
|---|---|---|---|---|---|---|
| identification | | | | | | |
| obtained from | | | | | | |
| subcultures | | | | | | |
| (biochemical or | | | Identification | | | |
| molecular | Tested | | | | | Unacceptable |
| techniques) | samples | Species | Group | Genus | Family | profiles |
| [symbol] phylococcus | | | | | | |
| S. aureus | 42 | 42 | | | | |
| S. epidermidis | 7 | 7 | | | | |
| S. haemolyticus | 7 | 6 | 1 (CNS group^{a}) | | | |
| S. hominis | 3 | 3 | | | | |
| S. warneri | 5 | 5 | | | | |
| S. lugdunensis | 2 | 2 | | | | |
| Micrococcus luteus | 5 | 3 | 1 (CNS group^{a}) | | 1 | |
| [symbol] eptococcacae | | | | | | |
| S. mitis | 6 | | 6 (pneumoniae/mitis group) | | | |
| S. pneumoniae | 6 | | 6 (pneumoniae/mitis group) | | | |
| S. gordonii | 4 | | 4 (oral streptococci group^{b}) | | | |
| [symbol] terococcus | 14 | 14 | | | | |
| faecalis | | | | | | |
| [symbol] am negative non | | | | | | |
| [symbol] menting bacilli | | | | | | |
| P. aeruginosa | 39 | 38 | | | 1 | |
| S. maltophilia | 20 | 18 | | | | 2 |
| A. baumanii | 15 | 15 | | | | |
| [symbol] terobacteriacae | | | | | | |
| E. coli | 20 | 19 | | | | 1 |
| E. cloacae | 18 | 13 | | | 5 | |
| Citrobacter group | 16 | 14 | | | 1 | 1 |
| freundii | | | | | | |
| K. oxytoca | 17 | 17 | | | | |
| K. pneumoniae | 20 | 18 | 1 (KES group) | | | 1 |
| P. mirabilis | 18 | 17 | | 1 | | |
| [symbol] influenzae | 2 | 2 | | | | |
| [symbol] aerobes | | | | | | |
| C. perfringens | 2 | 2 | | | | |
| F. necrophorum | 3 | 3 | | | | |
| B. fragilis | 1 | 1 | | | | |
| [symbol] albicans | 10 | 10 | | | | |
| [symbol] her Candida | 10 | 10 | | | | |
| [symbol] cies | | | | | | |
| [symbol] tal | 312 | 279 | 19 | 1 | 8 | 5 |

^{a}CNS group: coagulase negative Staphylococcus
^{b}Oral Streptococci group: group including all oral species of Streptococci excepted the S. milleri group [symbol]
indicates data missing or illegible when filed

Of the 307 interpretable spectra (98%), MALDI-TOF-MS allowed a good identification at the species, group, genus and family level in 89%, 6%, 0.4% and 2.6% of cases, respectively. It should be pointed out that MALDI-TOF-MS allowed differentiation of coagulase negative Staphylococci (CNS) from Staphylococcus aureus in 100% of cases.
2) Identification of Positive Blood Cultures from Patients
Among the 388 positive blood cultures included in this study, 373 were monomicrobial (table 2).

TABLE 2
Direct bacterial identification by MALDI-TOF-MS in monobacterial blood cultures from patients

| Species | | | | | | |
|---|---|---|---|---|---|---|
| [symbol] dentification | | | | | | |
| [symbol] btained from | | | | | | |
| subcultures | | | | | | |
| [symbol] iochemical or | | | Identification | | | |
| molecular | Tested | | | | | Unacceptable |
| techniques) | samples | Species | Group | Genus | Family | profiles |
| [symbol] hylococcus | | | | | | |
| S. epidermidis | 121 | 118 | 3 (group CNS^{a}) | | | |
| S. haemolyticus | 3 | 3 | | | | |
| S. hominis | 20 | 20 | | | | |
| S. pasteuri | 1 | 1 | | | | |
| S. capitis | 3 | 2 | 1 (CNS group^{a}) | | | |
| S. aureus | 43 | 42 | | | | 1 |
| S. lugdunensis | 1 | 1 | | | | |
| [symbol] crococcus luteus | 1 | 1 | | | | |
| [symbol] tococccae | | | | | | |
| S. pyogenes | 8 | 5 | 3 | | | |
| | | | (pyogenes/dysgalactiae group^{b}) | | | |
| S. mitis | 8 | | 8 (pneumoniae/mitis group) | | | |
| S. pneumoniae | 1 | | 1 (pneumoniae/mitis group) | | | |
| S. gordonii | 1 | | 1(oral streptococci group^{c}) | | | |
| S. pasteurianus | 1 | | | | | 1 |
| S. salivarius | 1 | | 1 (oral streptococci group^{c}) | | | |
| S. oralis | 2 | 2 | | | | |
| E. faecalis | 6 | 3 | | 2 | 1 | |
| E. faecium | 2 | 1 | | | 1 | |
| [symbol] negative non | | | | | | |
| [symbol] enting bacilli | | | | | | |
| P. aeruginosa | 20 | 17 | | | | 3 |
| S. maltophilia | 4 | 4 | | | | |
| Achromobacter | 2 | 2 | | | | |
| xylosoxydans | | | | | | |
| B. cenocepacia | 1 | | 1 (B. cepacia/cenocepacia | | | |
| | | | group) | | | |
| P. oryzihabitans | 1 | 1 | | | | |
| [symbol] robacteriacae | | | | | | |
| E. coli | 41 | 40 | | | | 1 |
| E. cloacae | 24 | 24 | | | | |
| Cirobacter group | 9 | 6 | | | 1 | 2 |
| freundii | | | | | | |
| E. aerogenes | 7 | 5 | 1 (KES group) | | 1 | |
| K. oxytoca | 6 | 6 | | | | |
| K. pneumoniae | 10 | 10 | | | | |
| P. mirabilis | 8 | 8 | | | | |
| S. marcescens | 2 | 2 | | | | |
| [symbol] monella enterica | 3 | 3 | | | | |
| [symbol] fluenzae | 1 | 1 | | | | |
| [symbol] bicans | 11 | 11 | | | | |
| [symbol] l | 373 | 339 | 20 | 2 | 4 | 8 |

CNS group: coagulase negative Staphylococcus pyogenes/dysgalactiaegroup: no differentiation between S. pyogenes and S. dysgalactiae
Oral Streptococci group: group including all oral species of Streptococci exepted the S. milleri group [symbol]
indicates data missing or illegible when filed

Using MALDI-TOF-MS as described in the material and methods section an interpretable identification was obtained in 98% of cases. These results were concordant with those obtained by classical methods at the species, group, genus/family levels in 91%, 5%, and 2% of cases, respectively.

In addition, 15 blood cultures from patients containing mixed bacteria were tested (table 3).

TABLE 3
Direct identification by MALDITOF MS of blood culture containing ≧2 germs

| | | | Identification | | |
|---|---|---|---|---|---|
| | Isolated | | General | Gram negative | Gram positive |
| Gram | microorganisms | Number | database | Bacilli database | Cocci database |
| Gram positive | E. coli | | | | |
| cocci and | E. faecalis | 1 | E. coli | E. coli | E. faecalis |
| Gram | S. epidermidis | | | | |
| negative | A. baumanii, | 2 | A. baumanii | A. baumanii | Streptococcus |
| bacilli | S. mitis | | | | group |
| | | | | | pneumo/mitis |
| | E. faecalis + | 1 | E. faecalis | 0 | E. faecalis |
| | E. coli | | | | |
| | S. aureus | 4 | S. aureus (4/4) | P. mirabilis (2/4) | S. aureus (4/4) |
| | P. mirabilis | | | | |
| | P. aeruginosa + | 1 | P. aeruginosa/ | P. aeruginosa | S. epidermidis |
| | S. epidermidis | | S. epidermidis | | |
| Gram | E. coli + | 1 | E. coli | E. coli | NA |
| negative | M. morganii | | | | |
| bacilli | E. coli | 1 | E. coli | E. coli | NA |
| | K. pneumoniae | | | | |
| | E. coli + | 1 | E. coli | E. coli | NA |
| | P. mirabilis | | | | |
| | K. pneumoniae + | 1 | KES^{a} | KES^{a} | NA |
| | E. cloacae | | | | |
| Gram positive | E. faecalis + | 1 | E. faecalis | NA | E. faecalis |
| cocci | S. aureus | | | | |
| | S. haemolyticus + | 1 | S. hominis | NA | S. hominis |
| | S. hominis | | | | |

^{a}Group Klebsiella-Enterobacter-Serratia
NA: non applicable

Using the database, either only one of the pathogens present in the mixture was detected, or two pathogens were detected at the same score. When Gram positive cocci and Gram negative bacilli were detected on the Gram staining, the identification was improved in 6 out of 9 cases by using a database containing species specific spectra of Gram positive cocci or Gram negative bacilli, respectively.

3) Identification of Positive Enrichment Fluids from Blood Cultures Flasks:
fluids grown in blood culture broths were included. The results are given in a below (table 4).

TABLE 4
Direct bacterial identification by MALDI-TOF-MS
in enrichment cultures from patients

| Species identification | | | | |
|---|---|---|---|---|
| obtained from subcultures | | | | |
| (biochemical or | tested | identification | | |
| molecular techniques) | samples | Species | Group | Genus |
| Graft conservation liquids | | | | |
| E. coli | 3 | 2 | 1 (Shigella/E. | |
| | | | coli)^{a} | |
| H. alvei | 2 | 2 | | |
| S. epidermidis | 7 | 7 | | |
| S. warneri | 2 | 2 | | |
| S. cohnii | 1 | 1 | | |
| Articular fluids, bone | | | | |
| ponctions, deep abscesses | | | | |
| S. aureus | 16 | 16 | | |
| S. epidermidis | 5 | 5 | | |
| S. capitis | 1 | 1 | | |
| S. lugdunensis | 1 | 1 | | |
| E. coli | 4 | 4 | | |
| E. cloacae | 1 | 0 | | 1 |
| P. aeruginosa | 2 | 2 | | |
| S. pyogenes | 1 | 1 | | |
| | 46 | 44 | 1 | 1 |

^{a}Shigella/coli: no differentiation between Shigella sp and E. coli

All spectra were interpretable, and the obtained identification was concordant to that obtained by classical methods at the species, group and genus levels in 96%, 2% and 2% of cases, respectively.

### 4) Results on Urines

10 urines were tested in 9 cases E. coli was identified and in 1 case a S. saprophyticus strain was identified.

The identifications were correct.

The method of the invention can be directly used on biological samples if the germs concentration is sufficient and the infection is a microbial infection.

### 5) Time to Diagnosis

It should be pointed out that each positive blood culture was treated as soon as it was detected positive. The time required between the Bact/Alert alarm and the germ identification, including Gram staining performed during the incubation of detergent, was 20 min.

### DISCUSSION

The sensitivity and accuracy of pathogen detection by MALDI-TOF-MS applied directly from Bact-Alert bottles was evaluated. This study enables a rapid (20 min) and reliable identification of the vast majority of microorganisms isolated in blood or fluids cultures. A rapid and accurate diagnosis diminishes the use of inadequate and broad-spectrum antibiotics, thereby improving outcome and reducing the potential development of resistance and possible side effects. Identification of microorganisms in blood cultures by MALDI-TOF-MS dramatically extends the influence of the result of the Gram staining on clinical management. In particular, among the Gram-negative bacilli, the differentiation of Enterobacteriacae from Pseudomonas or Acinetobacter only 20 min after the blood culture growth will allow a more appropriate treatment pending the results of susceptibility testing. Similarly, the possibility to obtain an immediate diagnosis of S. aureus is of major clinical consequence. Fast differentiation of S. aureus from CNS should help the clinician to discriminate a serious infection from a possible contamination.

### REFERENCES

1. Carbonnelle, E., J. L. Beretti, S. Cottyn, G. Quesne, P. Berche, X. Nassif, and A. Ferroni. 2007. Rapid identification of Staphylococci isolated in clinical microbiology laboratories by matrix-assisted laser desorption ionization-time of flight mass spectrometry. J Clin Microbiol 45:2156-2161.
2. Degand, N., E. Carbonnelle, B. Dauphin, J. L. Beretti, M. Le Bourgeois, I. Sermet-Gaudelus, C. Segonds, P. Berche, X. Nassif, and A. Ferroni. 2008. Matrix-assisted laser desorption ionization-time of flight mass spectrometry for identification of nonfermenting gram-negative bacilli isolated from cystic fibrosis patients. J Clin Microbiol 46:3361-3367.

1- A method for identifying germs in a liquid medium, comprising adding a membrane detergent to the liquid medium containing components of a host infected with germs, i.e. cellular components and proteins from the extracellular environment, so as to release the germs from these components without degrading them, and in that the germs that have grown in the liquid medium are analysed by MALDI-TOF MS.

2- The method of claim 1, comprising the steps of:
adding to the liquid medium a mild membrane detergent which does not damage the cell wall of prokaryotes, in order to separate the germs from the components of the infected host, in particular releasing intracellular germs, and to obtain spectra with reduced background noise, thus allowing easy identification of the germs;
recovering the germs from the liquid medium by differential centrifugation; and
analysing the germ pellet by MALDI-TOF MS, followed by comparing the results with germ databases.

3- The method of claim 1, wherein the mild membrane detergent is added to the liquid medium to a final concentration of at most 5%, more preferably of 0.5-5%, most preferably of 1%.

4- The method of claim 1, wherein the mild membrane detergent is selected in the group comprising saponin, n-octyl glucoside, n-dodecyl-glucoside, octanoyl-N-methylglucamide, decanoyl-N-methylglucamide, n-dodecyl-β-D-maltoside.

5- The method of claim 1, wherein the contact time between the liquid containing the germs to be analysed and the detergent is at most 8 min, more preferably 1-8 min, more particularly approximately 5 min.

6- The method of claim 1, wherein the mixture is centrifuged at 20,000 rpm at most, particularly 10,000-15,000 rpm, for 15 min at most, preferably for approximately 1-15 min, and more particularly for approximately 2 min.

7- The method of claim 1, wherein the supernatant is discarded and the pellet is taken up in distilled water and centrifuged at 20,000 rpm at most, particularly 10,000-15,000 rpm, for 15 min at most, preferably for approximately 1-15 min, and more particularly for approximately 2 min.

8- The method of claim 1, wherein the liquids used are blood cultures from patients positive for a particular germ, joint fluids inoculated or not in a liquid medium, urines or any other biological fluid inoculated or not in a liquid medium, for example cerebrospinal fluid, drainage fluid from drains or abscesses, provided the infection is suspected to be monomicrobial.
